(19) United States
(12) Patent Application Publication
(10) Pub. No.: US 2010/0063485 A1
(43) Pub. Date: Mar. 11, 2010

(54) DEVICE FOR THE CONTROLLED RELEASE OF A PREDEFINED QUANTITY OF A SUBSTANCE

(75) Inventors: Markus Thomas Johnson, Eindhoven (NL); Ian French, Hove (GB); Marc Wilhelmus Gijsbert Ponjee, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/439,704

(22) PCT Filed: Sep. 6, 2007
(86) PCT No.: PCT/IB2007/053596

§ 371 Date: Mar. 3, 2009

(30) Foreign Application Priority Data
Sep. 8, 2006 (EP) 06120360.0

(51) Int. Cl. A61M 31/00; A61M 5/00; B65B 3/04; B32B 38/00; B32B 38/10

(52) U.S. Cl. 604/890.1; 604/93.01; 141/2; 156/281; 156/235; 156/249

(57) ABSTRACT

Device for the controlled release of a predefined quantity of a substance and method for the production of a device for the controlled release of a predefined quantity of a substance. To realize a controlled delivery of a substance based upon a multiplicity of individual compartments, the reservoirs are formed in plastic substrates that allow the substance delivery device to be flexible and conformal with both internal and external body parts. The fabrication technology for the plastic drug release reservoirs is compatible with active matrix array technology, allowing control of delivery to be based upon active matrix principles. Applications are for controlled external drug delivery (patches), implantable drug delivery and oral drug delivery (electronic pill).

The present invention relates to a device for the controlled release of a predefined quantity of a substance. The present invention further relates to a method for the production of a device for the controlled release of a predefined quantity of a substance.

Accurate delivery of small, precise quantities of one or more chemicals into a carrier fluid are of great importance in many different fields of science and industry. Examples in medicine include the delivery of drugs to patients using intravenous methods, by pulmonary or inhalation methods or by the release of drugs from vascular stent devices. Examples in diagnostics include releasing reactions into fluids to conduct DNA or genetic analysis, combinatorial chemistry, or the detection of a specific molecule in an environmental sample. Other applications involving the delivery of chemicals into a carrier fluid include the release of fragrances and therapeutic aromas from devices into air and the release of flavoring agents into a liquid to produce beverage products.

Devices for the controlled release of a predefined quantity of a substance are generally known. For example, the US patent application US 2004/0034333 A1 discloses an implantable device for controlled delivery of a drug, the device including a microchip which have reservoirs containing the molecules for release. The microchip device includes a substrate, at least two reservoirs in the substrate containing the molecules for release and a reservoir cap positioned on or within a portion of the reservoir and over the molecules, so that the molecules are controllably released from the device by diffusion through or upon disintegration or rupture of the reservoir caps. Each of the reservoirs of a single microchip can contain different molecules which can be released independently. One drawback of the known device is that each reservoir is directly contacted to an electrode which is used to electrically break the seal layer or the cap by applying a current and to release the drug. One external electrical connection is required for each compartment or for each reservoir from which the drug is to be released. A weakness of the prior art system is that the seal layer or the cap with the external electrical connections form a rigid release mechanism which is delicate in handling.

It is therefore an object of the present invention to provide a device for the controlled release of a predefined quantity of a substance with an increased number of compartments wherein the fitting of the device to a body shape is enhanced.

The above objective is accomplished by a device for the controlled release of a predefined quantity of a substance and a method for the production of a device for the controlled release of a predefined quantity of a substance according to the present invention. The device for the controlled release of a predefined quantity of at least one substance comprises a matrix arrangement of compartments, the release of substance of each compartment being controllable by an active matrix and each compartment being closed by at least one release mechanism, wherein the active matrix is provided at least partially on a first substrate layer and the release mechanism is provided on a second substrate layer.

An advantage of the device according to the invention is that it is possible to realize a controlled substance or drug delivery system based upon a multiplicity of individual drug release compartments where both the active matrix and the release mechanisms for each compartment are realized on a substrate layer. The device advantageously does not comprise any breakable rigid parts and thus, for example, can be taped in a curve to a patient's arm or wrapped around a tumor, e.g. a cancer, within the body.

A further advantage of the present invention is that the control of delivery of a substance or a drug is based upon an active matrix principle. This is in contrast to the prior art systems where each compartment is directly connected to an electrical connection. By the use of an active matrix, it is feasible to release drugs from any of the large number of compartments of the order of 100-1,000,000 in a controlled manner. This is not feasible if every compartment where to be individually controlled by a dedicated control device as the costs and space required to incorporate such a control system would be prohibitive.

The active matrix, in the sense of the invention, is realized by electrically connecting each compartment or at least each release mechanism of a compartment (for example two electrodes associated or attributed to a compartment) via at least one active component to one of a plurality of selection lines and/or to one of a plurality of signal lines. The active matrix is realized by connecting at least one of the electrodes of the release mechanism of the compartment to the selection lines and/or the signal lines via an active electrical or electronic component. Such active components include especially transistors like switch transistors (FET-transistors (field effect transistors) and/or bipolar transistors) but may also comprise other devices such as diodes or MIM (metal-insulator-metal) diodes. A further advantage of the present invention is that thereby, applications as for example external drug delivery systems (patches), implantable drug delivery systems or oral drug delivery systems (electronic pill) are possible. A drug delivery system according to the present invention may be applied for delivery of a single drug but can be advantageously applied to a system where several different drugs are applied from the same array or the same device. In a preferred embodiment at least one transistor is attributed to each compartment. In an alternatively preferred embodiment of the present invention, a first transistor and a second transistor are attributed to each compartment. An advantage of using a transistor or transistors as active components in an inventive device is that it is possible to render the inventive device cost-effective and still relatively small because it is possible to realize transistors on very small surface areas of, e.g., a glass substrate. According to the invention, the use of one or a plurality of transistors provides for an enhanced specificity in selecting a compartment compared to directly connecting the release mechanism to the selection and/or signal lines. The use of one transistor as active component aims at reducing relatively the required size (e.g. needed surface area) of a compartment. The use of at least a first and a second transistor aims at enhancing the functionality of driving the compartment (e.g. current and/or voltage controlled drug release) or at enhancing the functionality of the device (e.g. including further functions at each compartment like memorizing whether the drug release has already occurred or not).

It is much preferred, according to the present invention, to use a thin film transistor as the transistor or as the transistors for each compartment of the device. This renders the device more cost-effective and it is possible to use lighter materials.

In a further preferred embodiment, the active component comprises a memory means. This is advantageous in order to provide an enhanced control possibility of the functionality of the inventive device.

In a preferred embodiment of the present invention, the release mechanism is a one time release mechanism. This means that the release mechanism is in some manner “destroyed” by applying a release signal above the threshold and the release mechanism is not re-usable. Thereby, it is possible to provide the release mechanism very cost-effectively and easy to manufacture. Nevertheless, the present invention also refers to a release mechanism which is closable once it has been opened (for the first time) and further on re-openable at least a second time. Such an embodiment employing a re-closable and re-openable release mechanism is less preferred because this usually implies higher costs. In a further preferred embodiment of the present invention, the release mechanism of the compartment is provided removable or disintegratable by means of applying an electrical potential between a first electrode and a second electrode. It is thereby possible to very easily and quickly control the release of the substance out of one of the compartments. It is further preferred, that the release mechanism is activated by means of an electro-chemical reaction or by means of heating the release mechanism, preferably by means of an electrical current. The device can be produced in a very cost effective manner and the release of the substance can be made more quickly and more accurate.

Further embodiments of the present invention are provided with a control unit for controlling the release of the substance. It is further preferred, that the number of compartments is at least 100, preferably at least 1,000, more preferably at least 10,000, still preferably at least 100,000 and most preferably at least 1,000,000 compartments.

In a preferred embodiment of the invention, the arrangement of compartments is located between the first substrate layer and the second substrate layer. Advantageously, the substance is captured between the first substrate layer and the second substrate layer and thus the compartment structure can be kept simple.

The release mechanism of each compartment is preferably connected to the active matrix through the compartment. More preferable, the release mechanism of each compartment is directly connected to the active matrix, in particular by a conductive layer of the release mechanism which extends from the second substrate layer through the compartment to the first substrate layer. It is thereby possible to provide a highly reliable contact. Alternatively preferred, the release mechanism of each compartment is connected to the active matrix indirectly by the substance in the compartment. This embodiment is advantageous for the use with an electrochemical release mechanism.

In a further embodiment of the present invention, the first substrate layer and the second substrate layer are provided on the same side of the arrangement of compartments, in particular the first substrate layer and the second substrate layer are adjacently arranged.

Advantageously, the release mechanism and the active matrix can be easily integrated, which facilitates the electrical contact between the active matrix and the release mechanism. The combination of the first substrate layer and the second substrate layer thus borders the compartment from one side and, more preferably, the substance is released, typically by rupturing of the second substrate layer, through the first substrate layer. Most preferably, the first substrate layer and/or the second substrate layer is provided as a thin film. The thin film second substrate layer can advantageously take over the function of the thin metal/dielectric layer of the prior art drug release device.

The first substrate layer and/or the second substrate layer is preferably flexible. The device is advantageously adaptable to different applications and is very robust.

In a further preferred embodiment, the first substrate layer and/or the second substrate layer is subjected to mechanical stress. In particular for a thin flexible first substrate layer and/or the second substrate layer the rupture of the release mechanism is advantageously enhanced. For example, polyimide as a substrate material, may have a coefficient of thermal expansion (CTE) between 3 ppm/K and 50 ppm/K, depending upon the choice of polyimide. Preferably, the coefficient of thermal expansion of the second substrate layer is different to the coefficient of thermal expansion of the first substrate layer. Additionally or alternatively, the coefficient of thermal expansion of the second substrate layer is different to a coefficient of thermal expansion of a further substrate layer. By this it is easily possible to introduce a tensile stress in the adjacent first substrate layer and second substrate layer, which will enhance rupture.

In a preferred embodiment the compartments (20) are arranged in a backing plate. The backing plate is of a simple structure which is produced and filled with the substance at low cost. The combined first and second substrate layers may advantageously be laminated to the backing plate to enclose the substance.

In a further preferred embodiment, the volume of the compartments is at least partly determined by the shape of the first substrate layer and/or the second substrate layer. The person skilled in the art understands that the compartments are at least partly composed of the first substrate layer and/or the second substrate layer. The substance is advantageously filled into the first substrate layer and/or the second substrate layer and the compartments are closed by laminating a completely unstructured backing plate to the first substrate layer and/or the second substrate layer. The simple unstructured backing plate allows an even more flexible device.

In another preferred embodiment the device is so flexible that it may be coiled up. A coiled up device is furthermore preferred. Coiled up devices can more easily be fitted into small compact spaces, including veins or arteries. Coiled up devices are, for example, made by providing a flat package and mechanically coiling it up and locking it into position by adhesives, bands or welding, or by deliberately introducing mechanical stress into the thin flexible first substrate layer and/or the second substrate layer in order to induce the layers to coil up.

In a still further preferred embodiment of the present invention, a first group of compartments is provided to contain a first substance and a second group of compartments is provided to contain a second substance. An advantage of the device according to the present invention is that a very flexible substance release mechanism can be implemented in the structure of the inventive device. For example, it is possible to provide compartments of different size, thereby being able to contain different volumes of the substance or substances to release. For example, if at a given moment a greater quantity of a substance is to be released, a device can be controlled accordingly and open a compartment having an appropriate size and hence an appropriate volume of the substance to be released. This is instead of releasing the same quantity of substance from a certain number of smaller compartments which would have the same effect. Of course, the release of an appropriate quantity of a substance out of one single compartment is easier to control and therefore makes the device according to the present invention smaller, more light weight and more cost effective. Accordingly, the first and second substance can be different or identical. Another way to improve the flexibility of releasing substances like drugs or the like is to provide several different substances or different mixtures of substances in different compartments on the device, the different compartments being of the same or of a different size. It is thereby possible to controllably release for example two different drugs alternatively during the day or during another time interval to the patient. Alternatively it is also possible to further enhance the flexibility of use of the inventive device for example by providing differently sized compartments as well as different substances in the differently sized compartments. It is preferred, according to the present invention, that the compartments of the first group and/or the second group comprise at least two different volumes. It is thereby also possible to have a first group of compartments having a first volume or containing a first quantity of a substance, a second group of compartments containing each twice of the first quantity, a third group containing four times of the first quantity and a fourth group of compartments containing eight times of the first quantity. Thereby flexibility of releasing one or more substances is even further enhanced.

The present invention also includes a method for the production of a device for the controlled release of a predefined quantity of at least one substance, the method comprising the steps of

providing a matrix arrangement of compartments,

providing an active matrix at least partially on a first substrate layer for controlling the release of substance of each compartment,

providing a release mechanism for each compartment on a second substrate layer,

filling the compartments with the substance,

laminating the first substrate layer and/or the second substrate layer to a backing plate.

The production method allows advantageously low cost fabrication of controllable drug release systems which are very robust and miscellaneously applicable.

The active matrix is preferably fabricated on a plastic or metal foil substrate, thus providing an advantageously thin film flexible first substrate layer. Alternatively preferred, the active matrix is manufactured on an auxiliary substrate and then transferred from the auxiliary substrate to a flexible substrate, which advantageously reduces the production cost. More preferable, the auxiliary substrate is coated with a thin and/or flexible substrate layer, the active matrix is manufactured on the coated auxiliary substrate and the thin and/or flexible substrate layer is subsequently released from the auxiliary substrate, in particular by using a laser device. Preferably, the released thin and/or flexible substrate layer forms a part of the release device.

In a further preferred embodiment of the production process, the first substrate layer and/or the second substrate layer is subjected to mechanical stress and, after releasing from the auxiliary substrate, the device coils up due to the mechanical stress. Alternatively preferred, the device is provided as a flat package, the flat package subsequently being coiled up and locked in the coiled up position. Coiled up devices can advantageously be fitted into small compact spaces.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

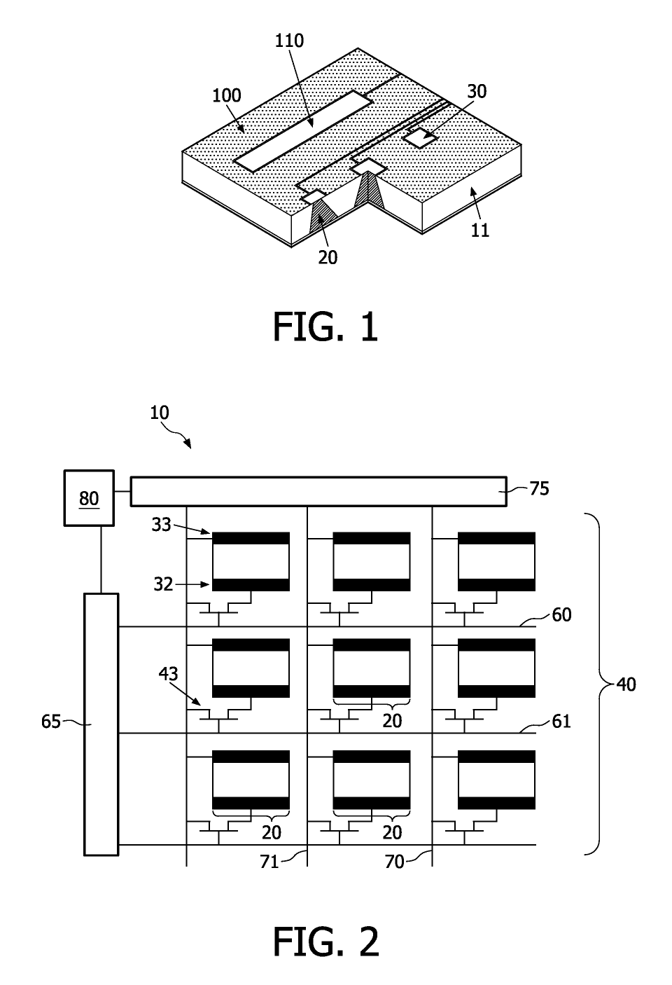

FIG. 1 illustrates schematically a device 100 according to the prior art showing a principle structure of a device of such a type.

FIG. 2 illustrates schematically a device according to the present invention.

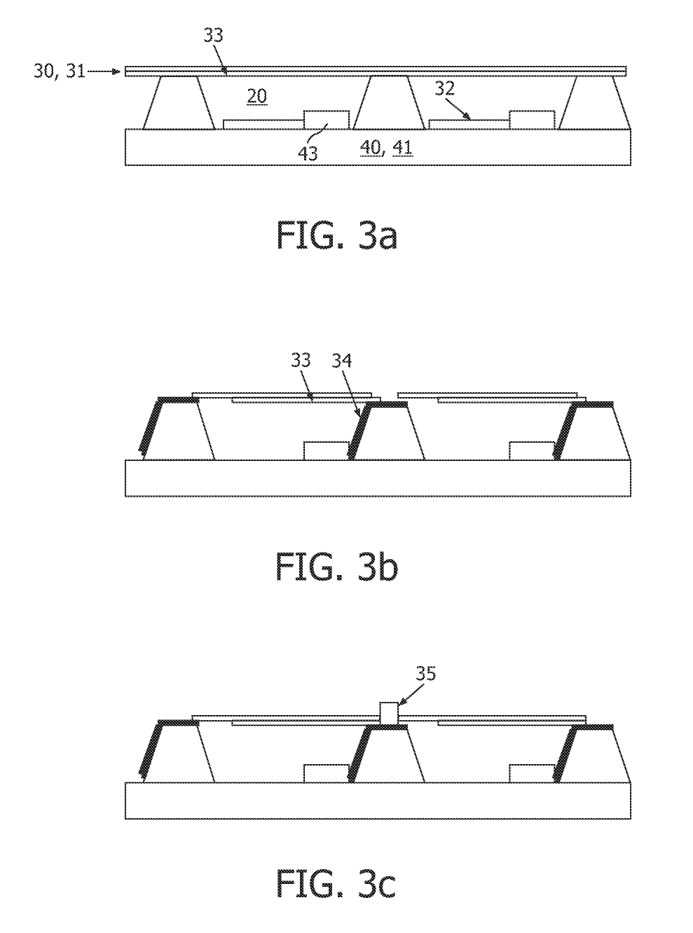

FIGS. 3a to 3c show schematical sections of three embodiments of the device according to the present invention.

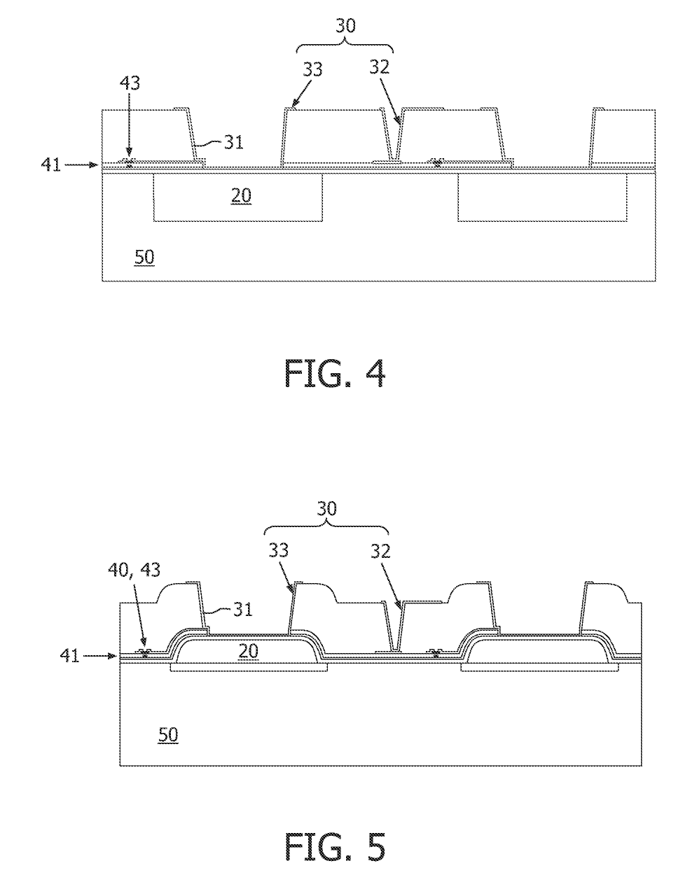

FIGS. 4 and 5 show further embodiments in schematical sections.

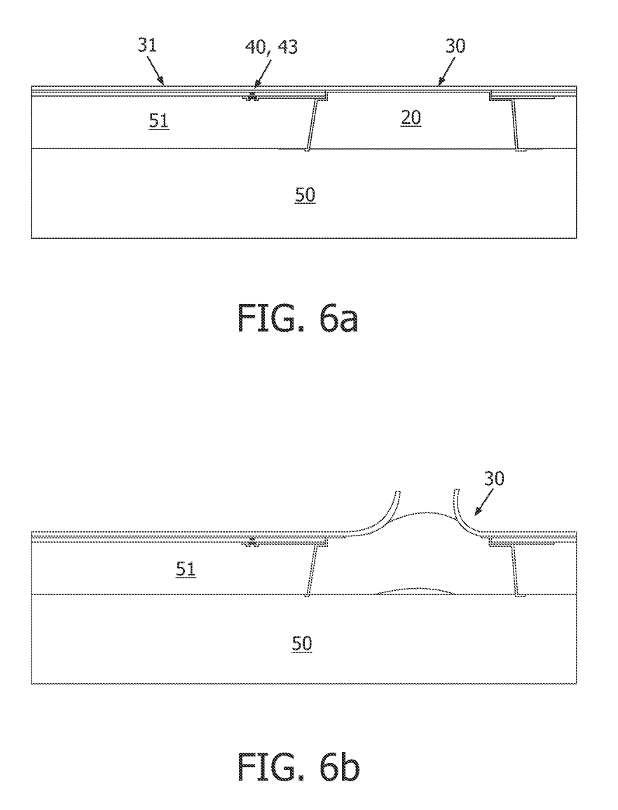

FIGS. 6a and 6b illustrates schematically the function of a compartment in a further embodiment.

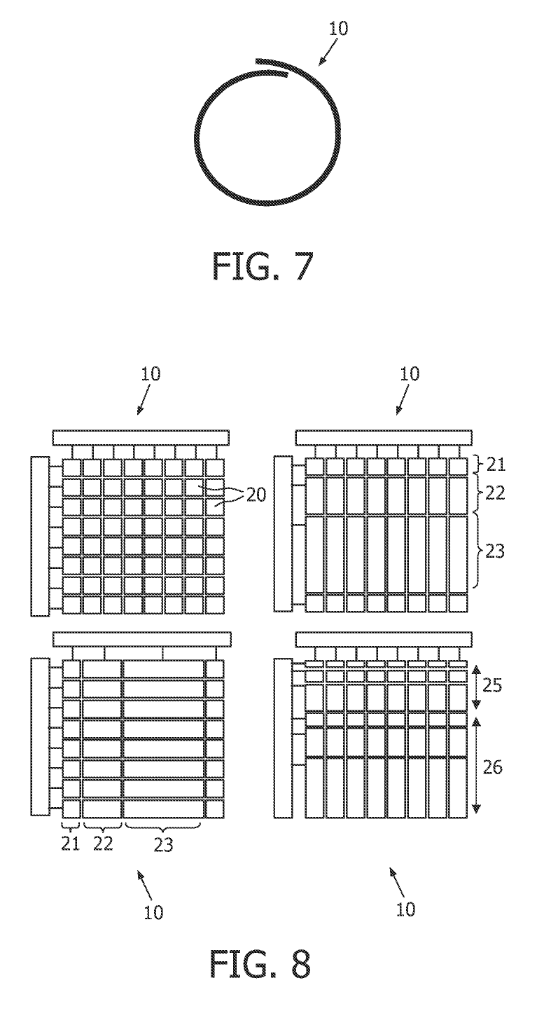

FIG. 7 illustrates schematically a further embodiment of the device.

FIG. 8 illustrates four different arrangements of compartments in a device according to the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described of illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIG. 1, a known device 100 according to the prior art is schematically shown. The known device 100 comprises a substrate 11 where a plurality of compartments 20 are located. The compartments 20 are closed by a release mechanism 30, especially a closure cap. It can further be seen from FIG. 1 that there exist electrode lines going to each of the compartments 20 or at least to or near to each of the release mechanisms 30. The connecting lines are not described with a reference sign in FIG. 1. The known device 100 further comprises an electrode area 110.

In FIG. 2 an inventive device 10 is schematically shown comprising a plurality of compartments 20 where only nine compartments 20 are shown. A compartment 20 (which is also called a reservoir in the following) is a container for a substance. Micro-electromechanical system methods, micro-molding and micro-machining techniques known in the art can be used to fabricate the device 10 together with the compartments 20 from a variety of materials. Examples of suitable substrate materials include glass, metals, ceramics, semiconductors, degradable and non-degradable polymers. Preferably, the substrates are the well known substrates for active matrix production of e.g. liquid crystal displays, namely glass, plastic films or metals. Bio-compatibility of the substrate material typically is preferred for in-vitro device applications. The substrate, or portions thereof, may be coated, capsulated, or otherwise contained in a bio-compatible material before use. The device 10 can have a variety of shapes for shaped surfaces and can be flexible. It can, for example, have a release side, i.e. an area having release mechanisms, that is planar or curved. The substrate of the device 10 may for example be in a shape selected from discs, cylinders, or spheres. In one embodiment, the release side can be shaped to conform to a curved tissue surface. This would be particularly advantageous for local delivery of a therapeutic agent to that tissue surface. In another embodiment the backside (distal to the release side) is shaped to conform to an attachment surface. The substrate may consist of only one material or may be a composite or multi-laminate material, that is, composed of several layers of the same or different substrate materials that are bonded together.

In the schematical illustration of FIG. 2 of the inventive device 10, the inventive device 10 comprises for each compartment 20 a first electrode 32 and a second electrode 33. Here, the first and the second electrodes 32, 33 are not directly electrically connected, i.e. they are substantially isolated from each other by e.g. a dielectric medium such as a fluid. This means, that the electrical resistance created by materials separating the first and second electrode 32, 33 from each other are from a sufficiently high resistivity that, regarding the applied voltages or potential differences, there is no substantial current flow between the first and second electrode 32, 33. The inventive device 10 further comprises the compartments 20 in the form of a matrix arrangement. Further, the inventive device 10 comprises a plurality of selection lines 60 and a number of signal lines 70. Here, the select and signal lines are shown in a mutually perpendicular alignment of rows and columns, whilst other matrix arrangements, such as on a hexagonal or triangular grid would also be possible, providing the select and signal lines are configured in mutually different orientations. For the sake of example, one specific selection line 61 out of the plurality of selection lines 60 is specifically shown in FIG. 2. Accordingly one specific signal line 71 out of the plurality of signal lines 70 is shown in FIG. 2. The specific selection line 61 and signal line 71 of FIG. 2 define the compartment 20 in the middle of the matrix arrangement of nine compartments 20 shown in FIG. 2. This means, that by selecting the specific selection and signal lines 61, 71, the compartment in the middle of the matrix arrangement is selected for being activated. The activation of the corresponding compartment is done by means of an active matrix 40 which, in the example of the inventive device 10 shown in FIG. 2, comprises one transistor 43 per compartment 20. The transistor is for example a FET-transistor, preferably a thin film transistor (TFT) as known from active matrix liquid crystal displays, having its gate terminal connected to the specific selection line 61 and having its source/drain terminal connected to the specific signal line 71.

Preferably, the thin film transistor is fabricated from any of the well known active matrix technologies as known from manufacturing of active matrix liquid crystal displays and other active matrix displays. These technologies include the amorphous silicon (a-Si) technology, low temperature poly silicon technology (LTPS), nanocrystalline Si technology, microcrystalline Si technology, CdSe technology, SnO technology, polymer or organic semiconductor based technology etc. In some cases only transistors of one polarity are available (e.g. a-Si provides only N-type transistors), whilst in other cases transistors of both polarity are available (e.g. LTPS provides n-type and p-type transistors). If an appropriate voltage level is applied to the specific selection line 61, the transistor switch will become conductive and thereby electrically connect the specific signal line 71 to the first electrode 32 (connected to the drain/source terminal of the transistor 43) of the compartment 20 in the middle of the matrix arrangement of compartment depicted in FIG. 2. This means, that the release mechanism 30 would be removed or activated by applying an appropriate electrical signal to the specific signal line 71. Of course, the function of the selection and signal lines 60, 70 (or the specific selection and signal lines 61, 71) can also be inverted, i.e. the signal lines 70 are connected to the gate terminal of the transistor 43 and the selection lines 60 are connected to the source/drain terminal of the transistor 43. Of course—as depicted in FIG. 2 —each compartment 20 is equipped with one transistor 43. The second electrodes 33 of each of the compartments 20 are commonly or in groups connected to further electrically conductive line. For the sake of clarity, this further electrically conductive line is not shown in FIG. 2.

For the sake of clarity, the release mechanism 30 is not depicted in FIG. 2. In contrast a first driver 65 (also called select driver 65) for driving the selection lines 60 is shown in FIG. 2 as well as a second driver 75 (also called central driver 75) for driving the signal lines 70. In the special example of FIG. 2, the gate terminals of the transistors 43 are connected to the select driver 65 (which can be provided as a standard shift register gate driver as used for an active matrix liquid crystal display) and the source terminals of the transistor 43 are connected to the central driver 75. Furthermore, a control unit 80 for controlling the release of the substance is also shown in FIG. 2. The control unit 80 controls the first and second driver 65, 75 for defining, by means of specific selection lines 61, 71, a specific compartment 20. The control unit 80 also controls the successive activation of different compartments 20. This means, that the control unit 80 for example controls the opening of the release mechanisms 30 of different compartments such that for example the concentration of a drug remain at an optimum therapeutic level during the course of a treatment. As the optimum concentration of the drug is variable from patient to patient, and during the course of the treatment, it is necessary that this drug delivery system is extremely flexible and providing an almost continuously variable dosage of the drug. Such a drug release system is possible to realize with the inventive device. Preferably the control unit 80 either has sensors for determining the actual level of the drug in the environment of the device 10 or the device 10 is coupled to such a sensor device (not shown) such that a signal from the sensor device signaling the control unit 80 to increase or decrease drug release results in an appropriate reaction of the inventive device, i.e. the control unit 80 activates the first and second drivers 65, 75 in order to increase or decrease the release of the substance inside the compartments 20.

As an example, if the drug delivery i.e. the opening of the release mechanisms 30, is based upon an electro-chemical reaction which breaks the seal of the compartment 20 or which breaks the release mechanism 30 of the compartment 20, and where a voltage of around 1 V is required to initiate the electro-chemical reaction. It is therefore possible to use a standard voltage data driver as used for e.g. active matrix liquid crystal displays. For example, one of the first and second electrode is provided as a cathode and the other electrode of the first and second electrode serves as an anode. The anode is defined as the electrode where oxidation occurs. Any conductive material capable of dissolving into solution or forming soluble ions or oxidation compounds upon application of an electric current or an electric potential (electrochemical dissolution) can be used for the fabrication of the anodes and cathodes. In addition, materials that normally form insoluble ions of oxidation products in response to an electric potential can be used if, for example, local pH changes near the anode cause these oxidation products to become soluble. Examples of suitable reservoir cap materials include metals such as copper, gold, silver, and zinc, and some polymers.

The inventive device 10 in the example shown in FIG. 2 works as follows: In the rest state, all selection lines 60 (also called select lines 60) are set to a voltage where the transistors 43 are non-conducting. In this case no release mechanism 30 is opened and therefore no substance or drug released. To release a substance or a drug out of one compartment 20, the transistors in the entire row of compartments 20 including the required compartment are switched into the conducting state (by e.g. applying a positive voltage). The voltage in the column where the compartment 20 to be activated is located is set to its release voltage (e.g. 1 V). This voltage is passed through the conducting thin film transistor to one of the first and second electrode 32, 33 of the selected compartment 20, resulting in drug release. The voltage in all other columns is held at a voltage which will not release the drug (this will be typically 0 V). After the drug is released, the transistors 43 in the selected row are again set to the non-conducting state, preventing further drug release.

It is also possible to release drugs or a substance or substances from more than one compartment 20 in a given line (or in a given row) simultaneously by applying a release signal (preferably a voltage) to more than one column in the array. It is possible to sequentially release drugs from compartments 20 in different rows by activating another one of the selection lines 60 (using the select driver 65) and applying a release signal (preferably a voltage) to one or more columns selection lines 70 in the array. The specific compartment 20 which is selected by the specific selection line 61 and the specific signal line 71 in FIG. 2 is located in the middle of the matrix arrangement of compartments 20. If the transistor 43 is conducting and the specific signal line activated, the voltage between the first and second electrode 32, 33 of the selected compartment 20 is then amounting for example to 1 V, thus initiating the drug release. The voltage in the other compartments 20 is held at a voltage which will not release the drug. After the drug or the substance is released, the selection line 60 and the signal line 70 are again set to 0 V which corresponds also to the rest state of the inventive device 10 thereby saving electrical power.

In one embodiment of the present invention it is also possible to release a drug or a substance from more than one compartment in a given row simultaneously by applying a release signal to more than one row, i.e. more than one specific selection line 61 in the array. Then different compartments 20 are simultaneously selected as being active, i.e. as being opened through removing the release mechanism 30 or by disintegrating the release mechanism 30. Accordingly it is also possible to simultaneously or sequentially release drugs from compartments 20 in different columns by activating a specific selection line 61 and applying a release signal to one or more columns in the array.

In another embodiment of the present invention, the drug delivery mechanism, i.e. the mechanism for opening the release mechanism 30, is based upon a heating effect, i.e. the heating of the release mechanism 30 breaks the release mechanism 30 of the compartment 20 which is selected. In this case, electrodes 32, 33 are electrically connected via the heating element, which could be any one of the known heating elements such as a resistive heater, peltier element etc.

When the release mechanism, i.e. the opening mechanism of the release mechanism 30 is provided as an electro-chemical reaction, the first or second electrode 32, 33 can, for example, be provided as a gold layer in the vicinity of the release mechanism 30. The other of the first and/or second electrode 32, 33 is for example another metallized electrode commonly connected. By applying a voltage between the first and second electrode 32, 33 a gold layer or gold cap acts as an anode in an electro-chemical reaction and is dissolved when a sufficiently high voltage is applied. After the electro-chemical reaction has taken place, the substance or drug inside the compartment 20 is free and allowed to diffuse away.

According to a feature of any of the described embodiments of the present invention, the device 10 can be packaged with a battery and a micro processor or a control unit to be completely self contained. Preferably the control unit 80 is integrated into the device 10 with the compartments 20.

The contents of the compartment 20 comprise essentially any object or material that needs to be isolated (e.g. protected from) the environment outside of the compartment 20 until a selected point in time, when its release or exposure is desired. In various embodiments, the compartment 20 contents comprise a certain quantity of molecules or of a specific substance or of a mixture of specific substances. Proper functioning of certain reservoir contents such as a catalyst or a sensor generally does not require the release of the compartment content. Rather, their intended function, e.g. catalyses or sensing, occurs upon exposure of the reservoir contents to the environment outside of the compartment 20 after opening of the closure cap 30. Thus, the catalysts molecules or sensing component can be released or can remain immobilized within the open compartment 20. Other compartment contents such as drug molecules often may need to be released from the compartment in order to pass from the device and be delivered to a site in vivo to exert a therapeutic effect on a patient. However, the drug molecules may be retained for certain in-vitro applications. The compartment 20 contents can include essentially any natural or synthetic, organic or inorganic molecule or mixture thereof. The molecules may be in essentially any form, such as a pure solid or liquid, a gel or hydrogel, a solution and emulsion, a slurry or a suspension. The molecules of interest may be mixed with other materials to control or enhance the rate and/or time of release of an open compartment 20. In various embodiments, the molecules may be in the form of solid mixtures, including amorphous or crystalline mixed powders, monolithic solid mixtures, lyophilized powders and solid interpenetrating networks. In other embodiments, the molecules are in liquid comprising forms, such as solutions, emulsions, colloidal suspensions, slurries or gel-mixtures such as hydrogels.

In FIGS. 3a to 3c, embodiments of a part of the device 10 are shown in a sectional view. The active matrix 40 with the transistors 43 is provided on a first substrate layer 41 which could be made on silicon, glass or plastic substrates. The drug release mechanism 30 (which for explanatory purposes will be considered to be a rupturing cap) is incorporated with a second substrate layer 31. The release mechanism 30 is electrically connected to the active matrix 40 on the first substrate layer 41. This is illustrated in FIG. 3 a-c, where in this case, the second substrate layer 31 and the release mechanism 30 is provided above the first substrate layer 41 with the active matrix 40, with the drug being enclosed in the resulting compartments 20, between the first substrate layer 41 and the second substrate layer 31. FIG. 3a depicts an embodiment of the device, showing indirect electrical contact through the drug. A connection through a conductive layer is depicted in FIG. 3b and with an additional conducting paste in FIG. 3c.

In the embodiment shown in FIG. 3a, the compartment opening is achieved by an indirect electrical signal from the active matrix 40, mediated by the drug to the release mechanism 30. This is in particular suitable if the release mechanism 30 is ruptured using the electrochemical release mechanism. In this case, all of the second electrodes 33 are held at a reference voltage (e.g. 0 V) and the release mechanism 30 is activated by applying a voltage to the individual first electrodes 32 on the first substrate layer 41 with the active matrix 40. The approach works best in combination with specific release mechanisms 30, such as the electrochemical release mechanism.

In FIG. 3b, the opening of the compartment 20 is achieved by a direct electrical signal from the active matrix 40, through an electrical connection 34 in the form of a via, to the release mechanism 30. Electrical contact could be achieved by, for example, extending the conducting layer 34 from the second substrate layer and through the compartment 20 to the first electrode 32 of the active matrix 40. The second substrate layer 31 of the release mechanism 30 is ruptured by passing a current through the release mechanism 30, using a resistive heating mechanism. In order to avoid the difficulty, that a part of the release mechanism 30 has to be deposited after the compartment 20 has been filled, the contact from the first substrate layer 41 to the second substrate layer may be realized by filling the connection 34 with a conducting paste 35 or glue 35, as also shown in FIG. 3c.

In FIGS. 4 to 6 embodiments of the inventive device 10 are depicted, wherein the release mechanism 30 is incorporated into the active matrix 40. The first substrate layer 41 and the second substrate layer 31 are adjacently arranged, which facilitates the electrical contact between the active matrix and the release mechanism. In addition, as the first substrate layer 41 and the second substrate layer 31 are adjacently arranged, it may be preferred during manufacturing of the device to realize a portion of the active matrix (for example the gate electrode, source and drain electrode, addressing or data lines etc.) directly on the same second substrate layer as the release device is formed. As a consequence, the substance is released through the first substrate layer 41 of the active matrix 40, in addition to being released through the (ruptured) second substrate layer. In order to achieve this, it is preferred that the first substrate layer 41 is thin and/or flexible, whereby it can take over the function of the thin metal/dielectric layer of the prior art release device.

Several approaches are known for realizing an active matrix 40 on a flexible first substrate layer 41, either by directly fabricating the active matrix 40 onto a plastic or metal foil substrate, or alternatively by transferring the active matrix 40 from an auxiliary (glass) substrate, on which it is manufactured, onto a flexible substrate. A further method is known as EPLaR (Electronics on Plastics by Laser Release) process, whereby the active matrix 40 is prepared on an auxiliary standard (glass) substrate coated with a thin flexible layer (such as poly-imide). The thin layer with active matrix 40 is subsequently released from the auxiliary glass substrate.

FIG. 4 illustrates an embodiment of the inventive device with release of the substance through the active matrix 40, i.e. through the first substrate layer 41, the compartments 20 being defined in a backing plate 50. The device is based upon the EPLaR process. The second substrate layer 31 comprises the release mechanism 30, for example in the form of an electrochemical reaction of a metal layer, for example a gold anode as second electrode 33, positioned above the thin flexible second substrate layer 31, and a gold cathode as first electrode 32. Optionally, the metal layer of the release mechanism 30 may be connected to a local current source on the active matrix 40, further electrodes 33, 32 connected together to allow current flow and the release mechanism embodied in the form of a resistive heating mechanism (not depicted). The connected first substrate layer 41 and second substrate layer 31 are laminated to the backing plate 50 in which the compartments 20 are provided (for example using an embossing or etching process). The compartments 20 in the backing plate 50 are first filled with the substance, for example using ink jet printing, before the connected first substrate layer 41 and second substrate layer 31 are laminated to it. Release of the substance occurs through the active matrix layer 40 in addition to being released through the (ruptured) second substrate layer when a compartment 20 is activated.

FIG. 5 shows a second example of device based upon the EPLaR process, where the substance is released through the active matrix 40 in addition to being released through the (ruptured) second substrate layer. The compartments 20 are defined in the first and second substrate layers 41, 31. The drug release mechanism 30, for example in the form of an electrochemical mechanism (depicted) or resistive heating (not depicted), is comparable to the one described in FIG. 4. The first and second substrate layers 41, 31 are connected to a simple, unstructured backing plate 50. The compartments 20 are first filled with the substance, before the simple backing plate 50 is laminated to it.

FIGS. 6a and 6b show a third example of device based upon the EPLaR process where the drug is released through the first and second substrate layers 41, 31 and illustrate the release of the substance from the filled compartment, FIG. 6a, after the compartment 20 has been opened, FIG. 6b. Again, the second substrate layer 31 comprises the release mechanism 30, in this example in the form of a resistive heating mechanism. The compartment 20 is formed in a plastic layer 51, closed by a backing plate 50. For small amounts of substance, the plastic layer 51 will be thin (4 to 100 μm) and polyimide could be used. For larger volumes of substance, the plastic layer 51 can be made thicker by the use of other plastics. For instance, SU-8 can be simply patterned to form wells in layers 100 to 200 μm thick. In this embodiment, the metal layer of the release mechanism 30 is situated on the same side of the release cap as the substance and, for this reason, may be covered by a thin, inert layer to prevent any possible interaction with the substance.

In FIG. 7, another embodiment is schematically depicted, wherein the device 10 is coiled up and thus can more easily be fitted into small compact spaces, including veins or arteries. Plastic coiled up devices can either be produced by providing a flat package and, subsequently, mechanically coiling it up and locking it into position by adhesives, bands or welding. Alternatively they are produced by deliberately introducing mechanical stress into the first and/or second substrate layers 41, 31, in order to induce the device 10 to coil up. For example, an active matrix TFT array is produced by the EPLaR process on a polyimide with a Coefficient of Thermal Expansion (CTE) of 50 ppm/K. The TFT array is flat after completion and coils up as soon as the laser process releases the polyimide from the auxiliary glass substrate. The coiling up is due to the difference in CTE between the polyimide and, for example, Silicium Nitrate, which has a CTE of approximately 3 ppm/K. The stress is introduced during the SiN deposition process, which typically takes place at 300° C. Alternatively, the stress may be induced between layers 31 and 51, which are thin flexible EPLaR layers but may comprise different polymers with different CTE's and/or thicknesses.

In FIG. 8, four different arrangements of compartments 20 within an inventive device 10 are schematically depicted. In a first embodiment of the device 10 (see FIG. 8 top left) all the compartments 20 are of the same size and provided in a matrix arrangement. The size of the compartments 20 defines a first quantity of a substance contained in the compartments 20. It is either possible that all compartments 20 contain the same substance or it is possible that in a first group (not shown) of the compartments 20, a first substance is located and that in a second group (not shown) of the compartments 20 a second substance is located.

In the second example shown in FIG. 8 (see FIG. 8 top right) an inventive device 10 is depicted where a first group 21 of compartments 20 has a predefine size, allowing to contain a first quantity of a substance. A second group 22 of compartments 20 comprises compartments 20 which are larger than the compartments 20 of the first group 21. Thus, the compartments of the second group 22 are for example able to contain a second quantity of a substance which is twice the first quantity. Of course every other ratio of the first and second quantities is also possible. A third group 23 of compartments 20 comprises compartments 20 which are able to contain a third quantity of a substance. The third quantity being for example twice the second quantity and four times the first quantity. Of course the third quantity can also be provided in a different ratio regarding the first and second quantity. By selecting specific compartments 20 out of the first the second or the third group 21, 22, 23 of compartments 20 it is possible according to the present invention to release a higher or lower amount or quantity of a substance out of the compartments 20 by means of just opening one single compartment 20. This has the advantage that the release of different quantities of the substance is possible to control very easily and with small efforts especially regarding the control unit 80.

In a third example of the inventive device 10 of the present invention depicted in FIG. 8 (see FIG. 8 bottom left) a matrix arrangement of compartments 20 with different groups 21, 22, 23 of compartments 20 is shown. In the third example the arrangement of compartments 20 is comparable to the arrangement of compartments 20 in the second example (FIG. 8 top right). In the third example the size of compartments in each row of the matrix arrangement is identical whereas the different groups of compartments are realized by changing the size of compartments 20 between different columns. In contrast, in the second example (see FIG. 8 top right) the compartments of each column are identically sized and the compartments of different rows are different.

In a fourth example of a matrix arrangement of the compartments 20 in an inventive device 10 according to the present invention, it is defined a first area 25 of compartments 20 which contains a first substance and there is defined a second area 26 of compartments 20 which contains a second substance.

By the examples given of different matrix arrangement of the compartments 20 of an inventive device, it is possible to have a high flexibility in dosing different quantities and/or different substances by means of the inventive device 10. By changing the size of the compartments 20 and hence the quantities of substances released, a more flexible drug delivery is possible with a smaller number of compartments. For example by providing compartments of sizes in the range of 1:2:4:8:16 etc. it is possible to provide a wide range of dosing a simultaneously opening one or more compartments 20 in a controlled manner. In the case of the delivery more than one type of substance (see example four of FIG. 8, bottom right) it is usual that different drugs have different dosing quantities. For this reason it will be preferred to have different sections or areas 25, 26 of the matrix array of compartments 20 with proportionally larger or smaller compartments 20 depending upon the drug to be delivered. This is preferably achieved by uniformly increasing this spacing between selection line 60 and/or signal line 70 in the array as is illustrated in FIG. 8 (bottom right) as this makes the best use of the available drivers 65, 75 and to reduce redundancies of elements included into the device 10. Depending on the complexity of the desired device 10, a memory or shift register is needed to keep the status of the used and still available compartments 20 updated. Such a memory device can advantageously be included into the control unit 80 of the device 10.

1. Device (10) for the controlled release of a predefined quantity of at least one substance, the device (10) comprising a matrix arrangement of compartments (20), wherein the release of substance of each compartment is controllable by an active matrix (40) and wherein each compartment is closed by at least one release mechanism (30), the active matrix (40) being provided at least partially on a first substrate layer (41) and the release mechanism (30) being provided on a second substrate layer (31).

2. Device (10) according to claim 1, wherein the release mechanism (30) is a one time release mechanism, the release mechanism (30) preferably being activated by means of an electrochemical reaction and/or by means of heating the release mechanism (30).

3. Device (10) according to claim 1, wherein the arrangement of compartments (20) is located between the first substrate layer (41) and the second substrate layer (31).

4. Device (10) according to claim 1, wherein the release mechanism (30) of each compartment (20) is connected to the active matrix (40) through the compartment (20).

5. Device (10) according to claim 4, wherein the release mechanism (30) of each compartment (20) is connected to the active matrix (40) by a conductive layer (34) of the release mechanism, the conductive layer extending from the second substrate layer (31) through the compartment to the first substrate layer (41).

6. Device (10) according to claim 4, wherein the release mechanism (30) of each compartment (20) is connected to the active matrix (40) by the substance in the compartment.

7. Device (10) according to claim 1, wherein the first substrate layer (41) and the second substrate layer (31) are provided on the same side of the arrangement of compartments (20).

8. Device (10) according to claim 1, wherein the substance is released through the first substrate layer (41).

9. Device (10) according to claim 1, wherein the first substrate layer (41) and/or the second substrate layer (31) is provided as a thin film.

10. Device (10) according to claim 1, wherein the first substrate layer (41) and/or the second substrate layer (31) is flexible.

11. Device (10) according to claim 1, wherein the first substrate layer (41) and/or the second substrate layer (31) is subjected to mechanical stress.

12. Device (10) according to claim 1, wherein a coefficient of thermal expansion of the second substrate layer (31) is different to a coefficient of thermal expansion of the first substrate layer (41) or the coefficient of thermal expansion of the second substrate layer (31) is different to a coefficient of thermal expansion of a further substrate layer.

13. Device (10) according to claim 1, wherein the compartments (20) are arranged in a backing plate (50).

14. Device (10) according to claim 1, wherein the volume of the compartments (20) is at least partly determined by the shape of the first substrate layer (41) and/or the second substrate layer (31).

15. Device (10) according to claim 1, characterized in that it is coiled up.

16. Device (10) according to claim 1, wherein a first group (21) of compartments (20) is provided to contain a first substance and a second group (22) of compartments (20) is provided to contain a second substance.

17. Device (10) according to claim 15, wherein the compartments of the first group and/or the second group comprise at least two different volumes.

18. Method for the production of a device (10) for the controlled release of a predefined quantity of at least one substance, comprising the steps of
providing a matrix arrangement of compartments (20),
providing an active matrix (40) at least partially on a first substrate layer (41) for controlling the release of substance of each compartment,
providing a release mechanism (30) for each compartment on a second substrate layer (31),
filling the compartments (20) with the substance,
laminating the first substrate layer (41) and/or the second substrate layer (31) to a backing plate (50).

19. Method according to claim 18, wherein the active matrix (40) is fabricated on a plastic or metal foil substrate.

20. Method according to claim 18, wherein the active matrix (40) is manufactured on an auxiliary substrate and then transferred from the auxiliary substrate to a flexible substrate.

21. Method according to claim 18, wherein an auxiliary substrate is coated with a thin and/or flexible substrate layer, the active matrix is manufactured on the coated auxiliary substrate and the thin and/or flexible substrate layer is subsequently released from the auxiliary substrate.

22. Method according to claim 21, wherein the first substrate layer (41) and/or the second substrate layer (31) is subjected to mechanical stress and, after releasing from the auxiliary substrate, the device (10) coils up due to the mechanical stress.

23. Method according to claim 18, wherein the device (10) is provided as a flat package, the flat package subsequently being coiled up and locked in the coiled up position.
